(19) United States
(12) Patent Application Publication
(10) Pub. No.: US 2009/0208409 A1
(43) Pub. Date: Aug. 20, 2009

(54) ENCAPSULATED NANOPARTICLES FOR COMPUTED TOMOGRAPHY IMAGING

(75) Inventors: Ronald M. Summers, Potomac, MD (US); Jianwu Xie, Rockville, MD (US); Celeste A. Roney, Gaithersburg, MD (US)

Correspondence Address: NATIONAL INSTITUTES OF HEALTH;C/O VENABLE LLP, P. O. BOX 34385, WASHINGTON, DC, 20043-9998, US

(73) Assignee: The Government of the United States of America, as represented by the Secretary, DHHS, Rockville, MD (US)

(21) Appl. No.: 12/371,472

(22) Filed: Feb. 13, 2009

Related U.S. Application Data
(60) Provisional application No. 61/064,086, filed on Feb. 15, 2008.

(51) Int. Cl. A61K 51/12; A61K 49/06; A61K 49/16; A61K 49/12; A61K 51/06; A61K 51/10; A61P 43/00

(52) U.S. Cl. 424/1.21; 424/9.1; 424/9.34; 424/9.3; 424/9.35; 424/1.49; 424/1.73; 424/9.36

(57) ABSTRACT

A detection agent, including a polymerized liposome, a lectin, and an imaging agent, can be administered to an animal for the detection of polyps in the lower gastrointestinal tract.

This application claims the benefit of U.S. Provisional Application No. 61/064,086, filed Feb. 15, 2008, which is incorporated by reference herein in its entirety.

### BACKGROUND OF THE INVENTION

The present invention relates to the use of detection agents, such as targeted nanoparticles including fucose-binding UEA-1 conjugated liposomes, that include an imaging agent, in conjunction with an imaging technology, to detect colon polyps. The present invention includes the use of such detection agents in the diagnosis of cancer.

Colon cancer or colorectal carcinoma (CRC) is the second leading cause of cancer related deaths in the United States, with more than 145,000 annual diagnoses [1]. Lifestyle choices such as diet, exercise, smoking, and alcohol consumption are contributing risk factors to CRC [2], however somatic and germline mutations also predispose to disease. For example, germline mutations in the tumor-suppressor adenomatous polyposis coli (APC) gene underlie the familial colorectal cancer called FAP (familial adenomatous polyposis). FAP manifests in adolescence, is characterized by thousands of adenomas in the colorectum, and advances to carcinoma if left untreated. According to Knudson's two-hit APC hypothesis, individuals affected by germline mutations (1^{st} hit) will develop FAP, with subsequent tumor progression after acquisition of the somatic mutation (2^{nd} hit) [3]. In this fashion, loss of heterozygosity (LOH) precludes disease.

The tumor suppressor gene APC, or adenomatous polyposis coli, acts along the adenoma-carcinoma pathway in the tumorigenesis of colorectal carcinoma (CRC). Genetic alterations of APC represent the earliest abnormality in the disease progression to carcinoma, which accounts for more than 55,000 annual moralities in the United States [4]. Aberrant crypt foci precede adenomas, developing through stages of polyposis and dysplasia, before invading the epithelial cells of the muscularis mucosae [5]. Despite manipulations of the APC gene in hereditary CRC, most cases are sporadic, and relate to epidemiological factors.

Other germline mutations, for example, of the mismatch repair genes (MMR) cause hereditary non-polyposis colorectal cancer, and also predispose to CRC [6]. Similar to APC genes, MMR genes are tumor suppressors requiring two genetic hits. However, a sub-category of MMR requires multiple genetic hits, resulting in tumors with an unstable genome (called microsatellite instability, MSI)[7]. Aaltonen et al have shown that tumors with the MSI characteristic provide markers for the MMR deficiency [8]. Other alleles implicated in familial CRC include AXIN2, POLD, and TGFβR2 [1]. Furthermore, numerous supplementary rogue genes are involved in the sequential progression from aberrant crypt proliferation through adenoma, carcinoma in situ, and metastatic carcinoma [9].

### SUMMARY OF THE INVENTION

A method according to the invention includes providing a detection agent and administering the detection agent to a section of lower gastrointestinal tract of an animal, such as a human, a small mammal, or another animal. The detection agent can include, optionally, a polymerized liposome or polymer, a lectin or antibody, and an imaging agent. The lectin or antibody can be conjugated to the liposome or polymer, or to the imaging agent. The imaging agent can be absorbed in the liposome or polymer. The method can further include permitting the lectin to bind to carbohydrate or glycoprotein or antibody to bind to antigen expressed by a gastrointestinal polyp or neoplasm, imaging the section of lower gastrointestinal tract, and identifying regions with elevated concentrations of the imaging agent. An elevated concentration of imaging agent can be a concentration of imaging agent that is greater than a baseline concentration of imaging agent. A baseline concentration of imaging agent can be, for example, a concentration of imaging agent associated with non-neoplastic, e.g., normal tissue, a concentration of imaging agent associated with a region of tissue at a different time, and/or a concentration of imaging agent associated with a different type of tissue. Imaging the section of lower gastrointestinal tract can include directing radiation to pass through the section of lower gastrointestinal tract of the animal and illuminate the imaging agent, and detecting radiation transmitted past, reflected by, emitted by, or modified by the imaging agent with a detector. For example, the imaging agent can modify the radiation by allowing only certain wavelengths of the radiation to pass through or be reflected by the imaging agent, allowing only radiation with a certain polarization to pass through or be reflected by the imaging agent, or changing the wavelength or polarization of radiation that passes through or is reflected by the imaging agent. The method can further include obtaining a radiograph from the detector, using the radiograph to develop a map of concentration of imaging agent, and using the map of concentration of imaging agent to develop a map of concentration of the carbohydrate and/or glycoprotein.

In a method according to the invention, the lectin includes Ulex europaeus agglutinin (UEA-1), the carbohydrate includes α-L-fucose, and the imaging agent includes a radiologic contrast agent and/or a fluorescent imaging agent.

In a method according to the invention, the map of concentration of carbohydrate or glycoprotein is used to develop a map of gastrointestinal polyps in the section of lower gastrointestinal tract, determine whether the section of lower gastrointestinal tract comprises a neoplasm, and/or determine whether the section of lower gastrointestinal tract comprises a carcinoma.

In a method according to the invention, the detection agent is administered orally in a pill, tablet, capsule, or other conveniently orally ingestible format.

In an embodiment according to the present invention, a detection agent includes UEA-1, another selective lectin, and/or an antibody and an imaging agent. The detection agent can further include a polymerized liposome, a polymerized vesicle, and/or a polymer. The UEA-1 or other selective lectin or antibody can be conjugated to the liposome, vesicle, polymer, and/or imaging agent. The imaging agent can be absorbed within the liposome, vesicle, or polymer.

### BRIEF DESCRIPTION OF THE DRAWINGS

FIG. 1A presents photographs from FITC lectin histochemistry.

FIGS. 1B-1 through 1B-3 present photographic images of APC^{Min/+} mouse tissue stained with FITC-UEA-1.

FIG. 2 presents an ex vivo multispectral optical image of an APC^{Min/+} mouse bowel incubated in FITC-UEA-1.

FIG. 3 presents a transmission electron microscope (TEM) image of polymerized liposomes showing their size (15-100 nm) and shape (spherical and tubular).

FIG. 4 presents a photograph of CT detection agent, UEA-1 conjugated liposomes, that include rhodamine fluorescent imaging agent, bound to polyps in C57BL/6J APC^{Min/+} mouse colon tissue. The image shows that UEA-1-conjugated polymerized liposomes target and bind APC^{Min/+} mouse colon polyps.

FIG. 5 presents an image of an adenomatous polyp growing into the lumen of an APC^{Min/+} mouse.

FIGS. 6A through 6D present images of histopathological slides of colon tissue showing α-L-fucose expression, as evidenced by UEA-1 binding.

FIGS. 7A through 7B present images from ex vivo Multispectral Imaging (MSI) of APC^{Min/+} mouse colons in the presence of FITC-UEA-1.

FIGS. 7C through 7D present optical images of APC^{Min/+} tissue.

FIG. 8 presents a cartoon of the mucin core backbone.

FIG. 9 presents a cartoon of the mucin core backbone with a lectin attached.

FIGS. 10A and 10B present transverse and sagittal CT images from an APC^{Min} mouse.

FIG. 11A presents a digital photograph of APC^{Min} tissue. FIG. 11B presents an image with H&E Stain of APC^{Min} tissue.

FIG. 12 presents a cartoon of an experimental design.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without parting from the spirit and scope of the invention. All references cited herein are incorporated by reference as if each had been individually incorporated.

Some diagnostic and screening approaches to colorectal carcinoma (CRC) use stool, for example, as a genetic surface marker [10]. Neoplasms secrete, leak, or exfoliate biomarkers [11], which then materialize in feces, and can be used for targeting in diagnostic or molecular imaging. Thus, stool testing is an attractive approach because it is both noninvasive, and reflective of the entire colorectum. Although the American Cancer Society has endorsed this technology as one with potential [12], not all surface markers have proven effective. For example, many members in the class of leaked markers lack specificity, and thus precision, due to nonspecific bleeding [10]. Leaked markers that have been tested as a screen for CRC include hemoglobin [13], calprotectin [14], lysozyme [15], and albumin [16].

Mouse models of colorectal carcinoma (CRC), including the adenomatous polyposis coli-multiple intestinal neoplasia (APC^{Min}) model of multiple intestinal neoplasia, can be employed in the analysis of various aspects of the cancer biology. We found it is a suitable animal model for diagnostic imaging studies of CRC in rodents, especially investigations which utilize computed tomography (CT).

The tumor suppressor gene APC (adenomatous polyposis coli) acts along the adenoma-carcinoma pathway in the tumorigenesis of CRC. Adenomatous polyps are benign neoplastic epithelia, however, those with a villous histology are clinically significant due to a high metastatic potential; they have a mucinous compounent, and may be a biological marker for CRC.

Mucins are high molecular weight glycoproteins with a heavy O-glycan concentration. Mucins line the surfaces of epithelial cells and aid the epithelia in homoeostatic and metabolic functions, such as digestion, absorption, and respiration. In addition, mucins, as a component of mucus, protect epithelia at interfaces with microorganisms, food, air, and water [18]. Mucins are found in cancer secretions, and on the surfaces of cancer cells, and as such, can be used as targets in cancer diagnosis [25]. Colorectal tissue is abundantly supplied with mucins throughout the normal and malignant mucosa; however, the adenoma to carcinoma transformation of cancerous cells alters O-glycan mucinous expression [26]. Kim et al. examined membrane glycopeptides in human colonic adenocarcinomas and normal mucosa [27]. The authors found reduced carbohydrate in tumor tissues, which reflected altered glycoprotein biosynthesis at the colonic membrane. Additional evidence of aberrant glycosylation, with emphasis on glycolipids, has been reported [28].

In a study designed to evaluate unglycosylated mucin in genetically deficient mice, Velcich et al [29] targeted Muc2, the most abundantly secreted gastrointestinal mucin. The authors report “intestinal tumor formation with spontaneous progression to invasive carcinoma,” upon loss of Muc2 activation. In addition, the Muc2−/− mouse model has reduced goblet cell formation [29], which is also seen in the aberrant crypt foci (ACF) of the clinical manifestation [30]. Finally, Limburg et al [31] have used a monoclonal antibody to the Muc1 protein to show 100% expression in colonic adenocarcinomas and 76% expression in adenomas, relative to 29% Muc1 expression by mucosa within 2 cm of the cancer margin, and 0% expression by normal mucosa greater than 2 cm of the cancer margin.

Lectins are plant and animal proteins with a strong glycoprotein affinity. Lectins are agglutinating carbohydrate-binding proteins capable of characterizing cell surfaces [32], since they function in cell-to-cell adhesion and recognition [33]. A few lectins have been shown to possess binding sites to colorectal mucosa [19-24]. Yonezawa et al. [23, 24] detected the α-L-fucose binding lectin Ulex europaeus agglutinin-1 (UEA-1) in human colorectal specimens of adenocarcinomas, adenomas, and polyposis coli, but not in the normal epithelium. Increased UEA-1 reactivity in polyposis patients with a familial history of large bowel carcinoma has been described [22]. Watanabe et al [34] report 83% positive rate of UEA-1 binding on the apical surfaces of human carcinoma cells, compared with 0% positive rate of UEA-1 binding on the apical surfaces of non-neoplastic mucosa adjacent to the carcinoma.

Ulex Europaeus Agglutinin I (UEA-1) can include the amino acids Asp, Ser, and Gly. It is a metalloprotein that can include 3 Ca⁺ ions and 1-2 Zn²⁺ or Mn²⁺ ions per 65 kDa. UEA-1 can be specific to blood group O. It can have a molecular weight of about 60-68 kDa. UEA-1 can be formed of two subunits, of 32 kDa and 29 kDa molecular weight. UEA-1 can have an isoelectric point (pI) of 6.0-6.1. UEA-1 can be specific to the α-L-fucose sugar.

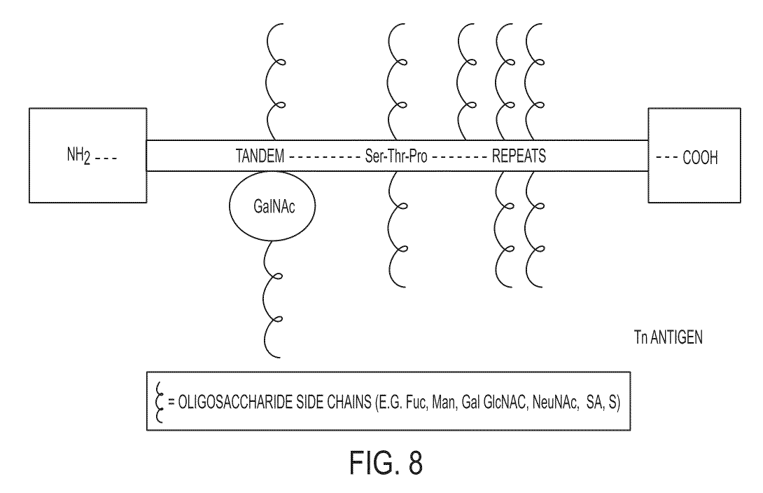

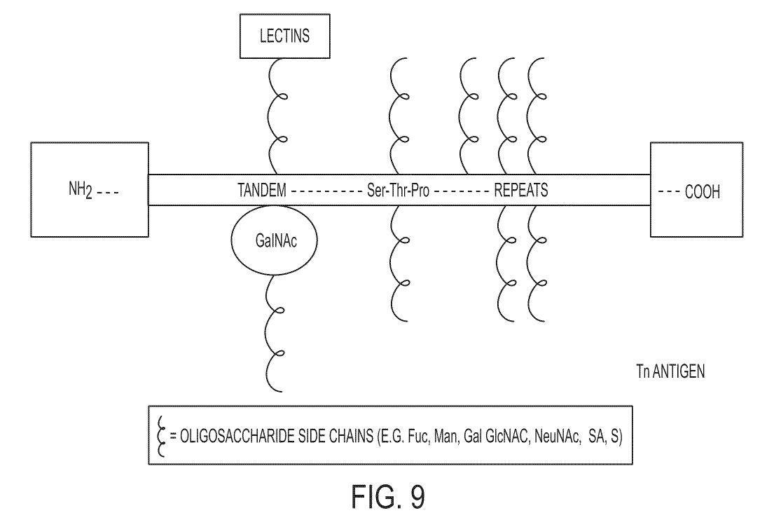

FIG. 8 presents a cartoon of the mucin core backbone. FIG. 9 presents a cartoon of the mucin core backbone with a lectin attached to an oligosaccharide side chain.

Recent literature has focused on stool as diagnostic markers in CRC detection [35, 36]. Shamsuddin et al [37] have tested mucin proteins as markers in CRC detection, although they have been shown as unlikely stool markers due to metabolism by bacterial enzymes inherently present in the colorectal tract [38]. However, exfoliated colonocytes have a potential for a unique specificity. For example, the exfoliation of colonocytes from neoplasms is unremitting, thus shed cells are in abundance, and can be obtained at various steps along the pathway to carcinoma [10]. Loktionov et al [39] isolated and quantified human DNA obtained from exfoliated colonocytes from the stool surface as a discriminatory marker, and Alquist et al [40] found the mucocellular layer overlying cancer cells 100-200 times more dense than the corresponding layer over normal epithelia. Furthermore, in a separate clinical study of CRC patients, Alquist et al [41] found mutant stool DNA to be as high as 24% of total recovered DNA. Finally, Boynton et al [42] have used molecular genetics (e.g. PCR) to analyze the integrity of long DNA fragments as markers in stool detection of CRC, meanwhile Dong et al [43] found multiple genetic targets, such as TP53, BAT26, and K-RAS, by screening stool DNA in clinical patients at risk for CRC. However, these techniques do not represent in vivo diagnostic imaging.

Adenomatous polyps are benign neoplastic epithelia, however, those with a villous histology are clinically significant due to a high metastatic potential. In colorectal carcinoma, polyps with villous histology may have a mucinous component. Mucinous polyps have an increased metastatic potential, and as such, mucin glycoproteins, and the carbohydrates of which they are composed, have a clinical significance in the diagnosis of metastatic disease.

An embodiment of the present invention includes the use of the legume lectin Ulex europaeus agglutinin I (UEA-1) to bind to the surfaces of colorectal carcinoma cells and to detect such colorectal cancer by in vivo imaging techniques. UEA-1 can bind the surfaces of adenomalous polyps in specimins of CRC from the APC^{Min} mouse model. An embodiment according to the present invention includes the use of α-L-fucose, overexpressed by colorectal polyps, as a biomarker. An embodiment includes the use of UEA-1 to bind to the carbohydrate α-L-fucose in targeting and detecting colon adenomas by imaging modalities, such as computed tomography.

In an embodiment of the present invention, a carbohydrate other than α-L-fucose that is overexpressed by neoplastic tissue, such as colorectal polyps, serves as a biomarker. UEA-1 or another lectin can bind to the carbohydrate to target and detect the neoplastic tissue by an imaging modality, such as computed tomography.

A detection agent according to the present invention can be used to image and detect colorectal polyps. In an embodiment, the detection agent includes a delivery agent, such as a polymerized liposome or polymer conjugated with a moiety capable of binding to a biomarker, such as a lectin or antibody, e.g., UEA-1. Alternatively, a micelle or a nanoparticle can be conjugated with a moiety capable of binding to a biomarker. The delivery agent can include one or more imaging agents. Examples of imaging agents are, a radiologic contrast agent, such as diatrizoic acid sodium salt dihydrate, iodine, or barium sulfate, a fluorescing imaging agent, such as Lissamine Rhodamine PE, a fluorescent or non-fluorescent stain or dye that can impart a visible color or that reflects a characteristic spectrum of electromagnetic radiation at visible or other wavelengths, e.g., infrared or ultraviolet, such as Rhodamine, a radioisotope, a positron-emitting isotope, such as ¹⁸F or ¹²⁴I (although the short half-life of a positron-emitting isotope may impose some limitations), a metal, a ferromagnetic compound, a paramagnetic compound, such as gadolinium, a superparamagnetic compound, such as iron oxide, and a diamagnetic compound, such as barium sulfate. The one or more imaging agents can be selected to optimize the usefulness of an image produced by a chosen imaging technology. For example, the one or more imaging agents can be selected to enhance the contrast between a feature of interest, such as a gastrointestinal polyp, and normal gastrointestinal tissue.

The detection agent can be placed into contact with tissue under examination, so that the detection agent adheres to a biomarker for colorectal polyps, such as an endogenous carbohydrates, e.g., α-L-fucose, which is overexpressed on the surface of colorectal polyps.

An imaging technology, such as colonoscopy, fluorescence microscopy, or computed tomography (CT) can then be used to detect the detection agent, and thereby identify the presence of the biomarker, and thereby, the colorectal polyps. The imaging technology can include virtual colonoscopy (also known as colonography), which can use, for example, a technique such as CT or MRI to image the colon. [44] For example, colonography can include the step of cleansing the bowel. Such a bowel cleansing step can include restricting a the diet of a subject (such as an animal, for example, a human, small mammal, or other animal) one to two days before the imaging procedure to clear liquids or low-fiber foods. On the day before and on the day of the imaging procedure, the subject can be required to drink a laxative, for example, a phospho-soda or polyethylene glycol solution. For example, before the imaging procedure, colonography can include the step of insufflating the gastrointestinal tract, e.g., the colon, with a gas, such as air, carbon dioxide, or nitrogen, to expand the gastrointestinal tract and render features of interest, such as colorectal polyps or neoplasms, more apparent. For example, in a CT scan, such insufflation can render polyps more apparent, because of the large contrast difference between air and soft tissue. Colonography can include performing scans with the subject in multiple positions, for example, in supine and prone positions. Altering the subject's position can help gas in the gastrointestinal tract, e.g., the colon, to shift and insufflate collapsed regions of the colon and allows for fluid to move and expose hidden polyps. For example, having a prone scan in addition to a supine scan can increase the sensitivity of detecting polyps. Colonography can further include tailoring the process of image acquisition by a technique such as CT or MRI. For example, polyps that are smaller than or equal to the thickness of a tomographic slice, such as a CT slice, may be affected by partial volume averaging and may not be detected by a radiologist or researcher. Thus, for colonography, the imaging technique can be performed with thin slices, e.g., with thin CT slices, to maximize the sensitivity of polyp detection. Furthermore, the parameters of the imaging technique can be set to achieve an optimum of decreased scanning time, decreased radiation exposure, and/or increased image quality. For example, a high pitch value can decrease the scanning time, a lower tube current can reduce radiation exposure, and a smaller slice thickness can improve image quality. Colonography can include the application of image processing techniques to increase the sensitivity of detection of polyps and neoplasms and reduce the incidence of false positive findings of polyps and neoplasms. For example, two-dimensional imaging can be used in conjunction with three-dimensional, e.g., three-dimensional fly-through, imaging. Translucency rendering, which can allow attenuation beneath the surface to be viewed, can increase specificity and decrease the time of interpretation by a radiologist. Having images read by more than one radiologist can improve the sensitivity for detecting polyps. Colonography can be used to stage a carcinoma. Computer-aided detection (CAD) systems can be used to increase the sensitivity of detection of polyps and neoplasms and decrease the time of interpretation by a radiologist. For example, a computer-aided detection (CAD) system can classify a surface based on its local shape by using a geometric curvature parameter. Principal (minimum and maximum) curvatures of the colonic surface can be assessed by a CAD system to distinguish colorectal polyps from haustral folds and other normal colonic structures. A CAD system can use textural features and/or attenuation in identifying targets such as polyps. A CAD system can apply filtering and smoothing techniques to improve the detection of small polyps. A CAD system can include improved display techniques that provide a reader, such as a radiologist, with an accurate spatial location of a feature in the gastrointestinal tract, e.g., the colon. A CAD system can include image processing techniques that diminish negative effects of artifacts and stool on image quality. A CAD system can segment the colon and generate colonic surface files. A three-dimensional endoluminal viewer can use such surface files in combination with images from a CT scanner to visualize anatomical landmarks and features such as polyps within the colon. Colonography can include a method of a positioning technique to identify longitudinal and circumferential positions in the gastrointestinal tract, e.g., the colon, which can, for example, facilitate the matching of a polyp or neoplasm identified in a supine scan with a polyp or neoplasm identified in a prone scan. For example, the detection of anatomic landmarks in the gastrointestinal tract, e.g., the colon, can allow for registration of supine and prone datasets that have been obtained and/or for earlier and later datasets that have been obtained. Such a method of registration can reduce the number of false positive indications of polyps or neoplasms. Such positioning and/or registration techniques can be incorporated into a CAD system. For example, a positioning technique can use the three smooth muscle bands that run longitudinally along the colon wall, the teniae coli. Identification of the teniae coli can allow for a centerline to be computed and a circumferential position of the polyp in the colon to be identified. The centerline can be used by a radiologist to determine relatively how far along the colon the polyp is located. A circumferential coordinate system can be established in reference to one of the teniae bands. For example, virtual colonoscopy (colonography) has been successfully used in analyzing the colon of a mouse scanned with microCT.

For example, example, electronic stool and fluid subtraction can allow for stool and fluid that has been treated with an oral contrast solution, for example, barium or iodine-based solutions or pills, to be removed from processed CT images. Such techniques can enable the CAD system to detect hidden polyps and reduce false positives. Such techniques may allow CAD to be performed without requiring the subject to take laxatives or adhere to a liquid diet. In certain cases, it may be useful to administer an oral contrast solution, e.g., barium or iodine-based solutions or pills, in conjunction with a detection agent according to the present invention. Such oral contrast solutions can adhere to polyps and villous polyps. An oral contrast solution may be useful for obtaining an image of parts of the gastrointestinal tract with normal tissue, to serve as a baseline against which images of suspected polyps or neoplasms can be compared.

For identifying polyps and neoplasms in the gastrointestinal tract, several techniques can be applied in conjunction. For example, optical colonoscopy can be used with CT imaging, or MRI imaging can be used with CT imaging.

A study was performed with 216 patients. 338 polyps were identified on CT colonography. 92 percent of polyps did not touch a contrast pool. 46 percent of the polyps not touching a contrast pool had adherent contrast. Of the polyps with a villous component, 77 percent had adherent contrast. Of the polyps without a villous component, 43 percent had adherent contrast. Thus, contrast material preferentially adhered to villous polyps. The adherent contrast may be a marker for the clinical significance of polyps. Oral contrast may adhere preferentially to the abnormal mucus made by villous polyps.

[symbol]

For example, dysplastic cells of colorectal polyps have a greater expression of the α-L-fucose component of the corresponding mucin than normal cells. Therefore, the α-L-fucose overexpressing polyps can be targeted by with UEA-1 conjugated liposomes that bind to α-L-fucose and the polyps. For example, a detection agent, such as UEA-1 conjugated liposomes, can be used in the field of small animal detection of colorectal carcinoma.

At present, patients undergoing colonoscopy are subjected to drinking 1 Liter of a poor-tasting oral contrast solution 24 hours prior to CT imaging. Additionally, some patients may be given an enema. The purpose of this solution is to cleanse the colon, by evacuating the bowels, and to provide the necessary contrast for imaging. Cleansing the colon in this manner is uncomfortable for the patient, as well as inconvenient; patients normally have to adjust their schedules so that they are continuously near a restroom. Furthermore, many patients do not adhere to colonoscopy schedules because of negative connotations associated with the routine of the present technique of cleansing.

By contrast, use of a detection agent according to the present invention may render such uncomfortable colon cleansing unnecessary. For example, a detection agent according to the present invention can be administered in a pill format. The detection agent may coat the surfaces of the polyps even in the presence of residual fecal matter in the uncleansed colon. The polyps would then be identifiable despite the presence of the residual fecal matter. Only a smaller subset of patients with polyps would need to undergo rigorous bowel cleansing and colonoscopic polypectomy. Such a method of administration should provide more comfort to the patient, and should increase patient compliance. Therefore, use of a detection agent according to the present invention can advance the means by which detailed in vivo colon information is obtained.

Radiation includes wave and particle phenomena that propagate through space and/or matter. For example, electromagnetic radiation, such as radio frequency, microwave, infrared, visible light, ultraviolet, X-ray, and gamma radiation, is a form of radiation. For example, sound, such as infrasound, audible sound, and ultrasound is a form of radiation. For example, alpha and beta radiation is a form of radiation.

A detector can detect one or more forms of radiation and transform the radiation into a signal, e.g., an electrical or light signal, that can be sensed by a human operator or can be further transformed, e.g., by an electronic circuit or computer. A radiograph includes an image produced from the detection of radiation by a detector. An image can be in one-, two-, or three-dimensions of space or can represent a higher dimensional object by a set of image slices of a lower dimension. For example, an image can include a set of two-dimensional image slices that represent a three-dimensional object. In this text, a radiograph includes images produced from the detection of all forms of radiation, including visible light.

Detectors used in producing radiographs can form part of an imaging technology. For example, photography can use a chemical detector, e.g., film, video camera tube, or solid state detector, e.g., charge coupled device image sensor to detect light and form an image of a sample. The image formed may represent light reflected by, transmitted through or past, or emitted by, e.g., upon stimulation of a sample with a form of radiation or energy, a sample. For example, X-radiography can use film or solid state detectors to detect X-rays and form an image of a sample. For example, computer tomography (CT) can use solid state detectors to detect X-rays and form an image of a sample, e.g., a three-dimensional image or a set of two-dimensional image slices that represent a three-dimensional object in a sample. For example, positron emission tomography (PET) can use one or more detectors to detect gamma radiation released by the annihilation of a positron emitted by a radioisotope and form an image of a sample. For example, single photon emission computed tomography (SPECT) can use one or more detectors to detect gamma radiation emitted by a radioisotope and form an image of a sample. For example, magnetic resonance imaging (MRI) can use one or more detectors to detect radio frequency radiation and form an image of a sample. For example, ultrasonic imaging can use one or more detectors to detect ultrasound and form an image of a sample.

A map can include an image that shows the distribution and/or form of one or more features over at least one spatial dimension. For example, a map can show the distribution of feature over a plane, over a three-dimensional volume as projected onto a plane, or over a three-dimensional volume. For example, a map of gastrointestinal polyps can show polyps in outline or as shaded, while not showing other features of the gastrointestinal tract.

A map of concentration can include an image that shows the distribution of concentration of a quantity over at least one spatial dimension. For example, a map of concentration shows the distribution of a quantity over a plane. For example, a map of concentration shows the distribution of a quantity over a three-dimensional volume as projected onto a plane. For example, a map of concentration shows the distribution of a quantity over a three-dimensional volume.

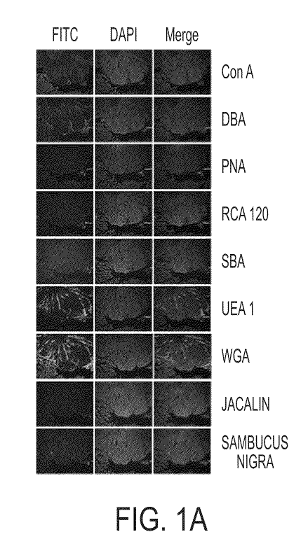

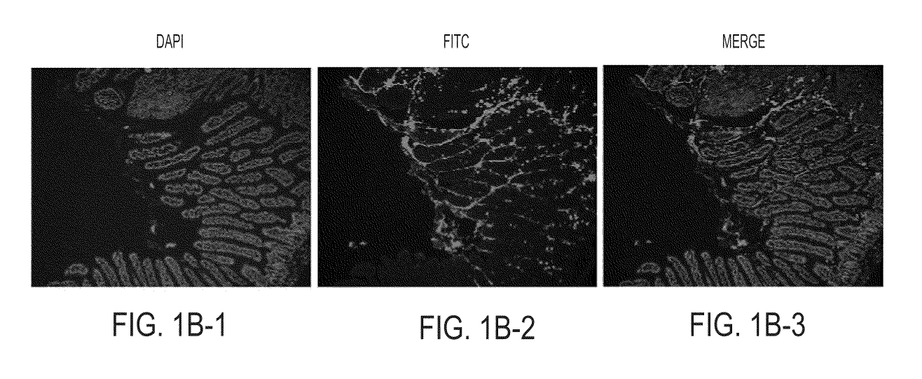

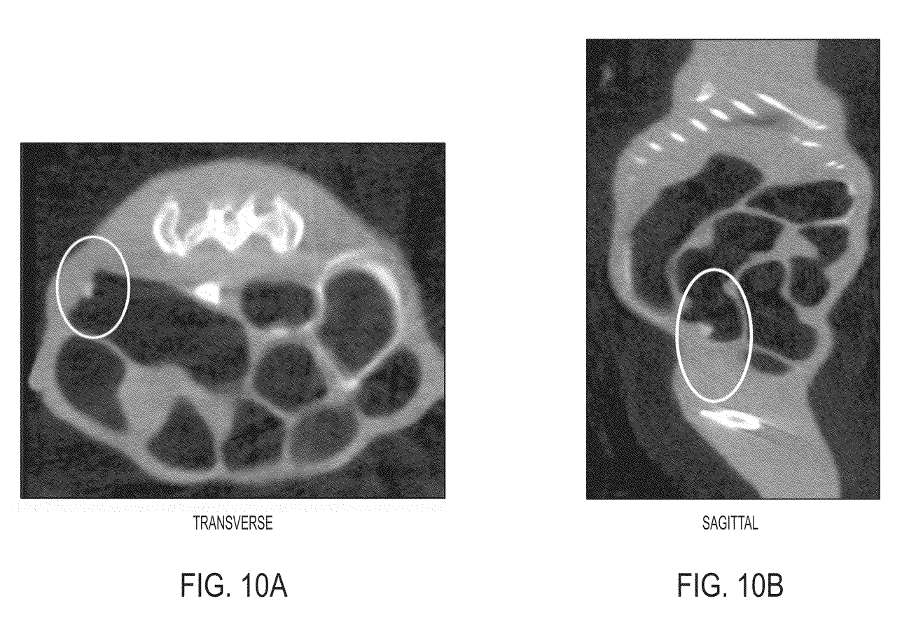

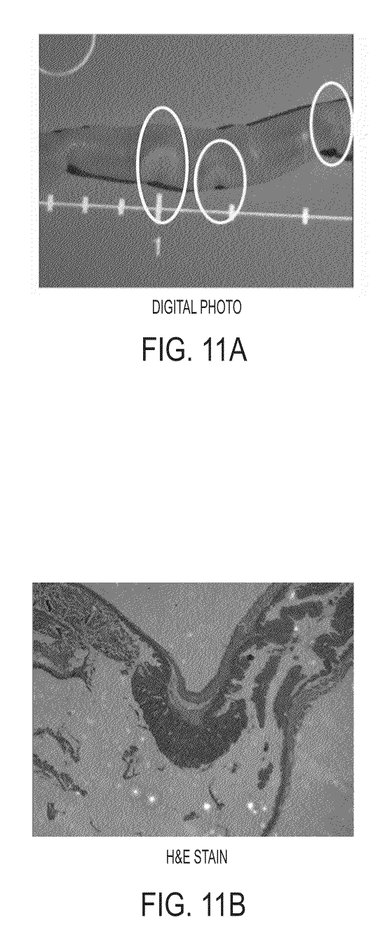

The bowels can be imaged using computerized tomography, for example, micro-computerized tomography. For example, the APC^{Min} (an APC gene truncation mutation) transgenic mouse model can be used. The bowels of the mice can be prepared by administering a solution of 20% Phosphosoda and 20 μg/mL Dulcolax at a dosage of 5 μL of solution per gram body weight of a mouse. A contrast agent of 30% Barium and 30% Gastroview can be administered at a dosage of 4 μL of contrast agent per gram body weight of a mouse. The colon can be insufflated with 3 cc of room air. The bowel can be imaged with micro-computerized tomography, for example, using the Siemens Imtek Micro CAT II. The images can be verified with histopathology. FIGS. 10A and 10B present transverse and sagittal images, respectively, of the APC^{Min} model. FIGS. 1A and 11B present a digital photo and an image with H&E stain, respectively. A limitation of imaging with computerized tomography can be that polyps are not always visible after administration of a Gastroview/Barium contrast agent.

### Example 1

### FITC Lectin Histochemistry

Nine different fluorescein (FITC)-labeled lectins were commercially purchased from Vector Laboratories (Burlingame, Calif., USA). The FITC-lectins used included: Concavalin A (ConA), Dolichos biflorus agglutinin (DBA), Peanut agglutinin (PNA), Ricinus communis agglutinin (RCA), Soybean agglutinin (SBA), Ulex europaeus agglutinin (UEA), Wheat germ agglutinin (WGA), Jacalin (Jac), and Sambucus nigra agglutinin (SNA).

Parafinized intestinal tissue sections (N=13, containing adenomatous polyps) were obtained from 8 weeks old heterozygous male mice of the strain C57BL/6J APC^{Min/+} (The Jackson Laboratory, Bar Harbor, Me.,) with 10% high fat diet. Paraffin sections were deparaffinized in three changes each of xylene, 100% ethanol (EtOH), 75% EtOH, 50% EtOH, and water. The final wash solvent was PBS. The sections were incubated in two antibody blocking solutions for 10 mins and 30 mins, respectively. These bowels excised from C57BL/6J APC^{Min/+} mice were used as a model of colorectal carcinoma.

Lectin staining for carbohydrate expression was performed overnight at 4° C. The FITC-UEA-1 lectins were diluted 1:500, using antibody diluent, to a final concentration of 5 μg/mL. The tissue sections were counterstained with DAPI, (2-(4-amidinophenyl)-1H-indole-6-carboxamidine), and the slides were examined under a fluorescence microscope.

The nine fluorescein-labelled lectins were used to analyze glycoprotein expression in the adenomatous polyps of the bowels excised from C57BL/6J APC^{Min/+} mice. The FITC-lectin histochemistry results are shown in FIGS. 1A and 1B. In FIG. 1A, the polyp is represented as the protruding mass of tissue occupying the majority of the image. Normal cells are represented around the bottom periphery of the image as long tubular cells. The tissues stained with WGA and DBA showed good secretory mucin expression on the polyp surface, but both also expressed on the normal surrounding mucosa. When the tissue stained with SBA was analyzed by fluorescence microscopy, mucin expression by goblet cells was identified, however, the expression was minimal; thus, glycoprotein expression was likewise minimal. Tissues stained with Jacalin and SNA showed polyp mucin expression, but not over-expression when compared with normal cells. Tissues stained with PNA and RCA did not show polyp mucin expression, and tissue stained with ConA showed over-expression of glycoprotein by normal cells. The tissue stained with UEA-1 showed UEA-1 strongly binds to polyps and not to adjacent normal mucosa. This result indicates a potential to use UEA-1 to differentiate polyps and normal mucosa.

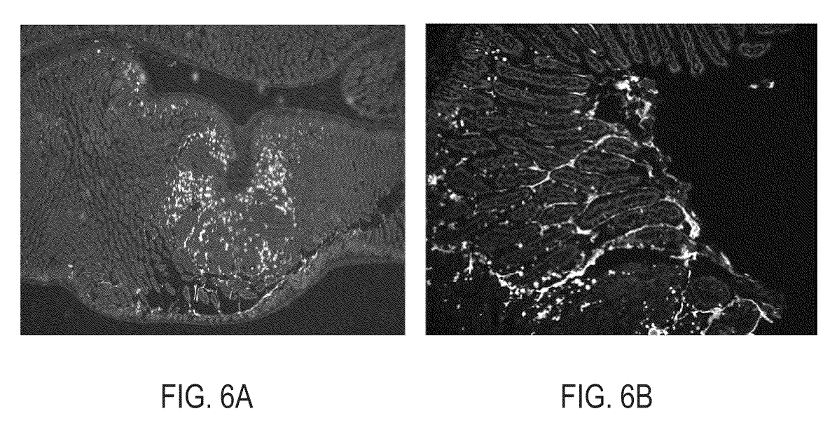

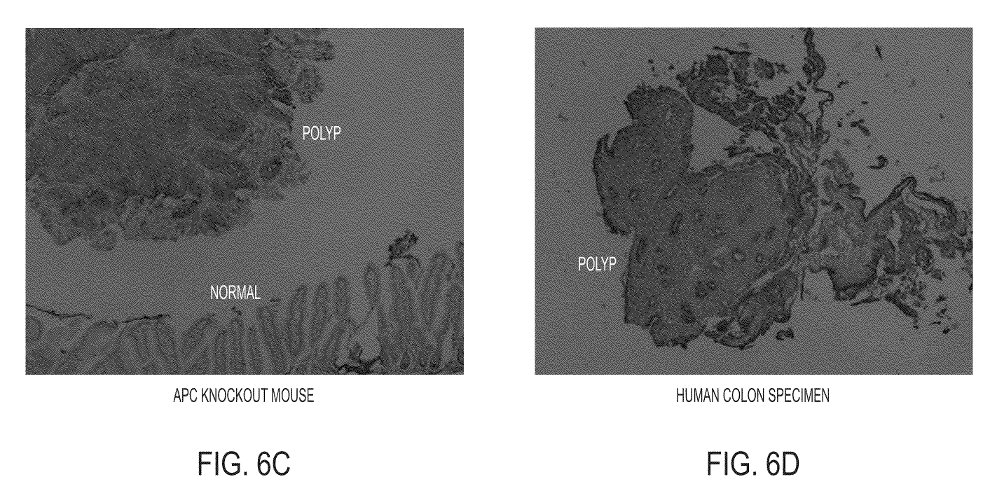

FIGS. 6A through 6D present images of histopathological slides of colon tissue showing α-L-fucose expression, as evidenced by UEA-1 binding. FIGS. 6A and 6B show that FITC-UEA-1 binds to the surfaces of polyps and not normal mucosa (the bright regions of FIGS. 6A and 6B indicate polyps, the darker regions of tissue indicate normal cells). Biotin-UEA-1 binds to the surfaces of the polyps on APC^{Min+/} (FIG. 6C) and human colon cells (FIG. 6D).

Thus, UEA-1-recognized fucose sequences were determined to be a probable marker for polyps in the APC^{Min/+} mouse. FIG. 1B shows the fluorescence microscopy results of the APC^{Min/+} tissue stained exclusively with FITC-UEA-1. The H & E stain, which is located above the image labeled FITC, shows the protruding polyp occupying the majority of the image space. The long, tubular cells at the bottom periphery of the image are normal cells. From the merged FITC (showing fluorescence by α-L-fucose-UEA-1 bond) and DAPI (showing DNA) images, it is clear that the polyp over expresses α-L-fucose; this compares with no carbohydrate expression by the normal cells. Much of the carbohydrate was expressed on the surface of the polyp, which can be used in developing targeting designs for diagnostic imaging.

### Example 2

### Ex Vivo Multispectral Optical Imaging

Excised small and large bowels (N=4) of male C57BL/6J APC^{Min/+} mice were commercially purchased from The Jackson Laboratory (8 wks old with 10% high fat diet) and stored at −80° C. until use. The bowels were thawed to room temperature (RT, 15-20 mins), cut along the longitudinal axis, and preserved in formaldehyde (RT, 15 mins) before the commencement of staining.

The bowels were incubated in 15 mL conical tubes in two antibody blocking solutions for 10 mins and 30 mins, respectively. After blocking, the bowels were incubated, with constant gentle shaking, in 5 μg/mL FITC-UEA-1 for 30 mins and followed by 3 times 5 mins PBS washing. Optical imaging was performed by using Maestro™ In-Vivo Multispectral Imaging System (CRI, Inc., Woburn, Mass.). During image acquisition, FITC was characteristically excited at 494 nm although emission data were collected from 500 to 950 nm including data from FITC's characteristic emission at 518 nm. The final reconstructed image excluded the scatter of background light.

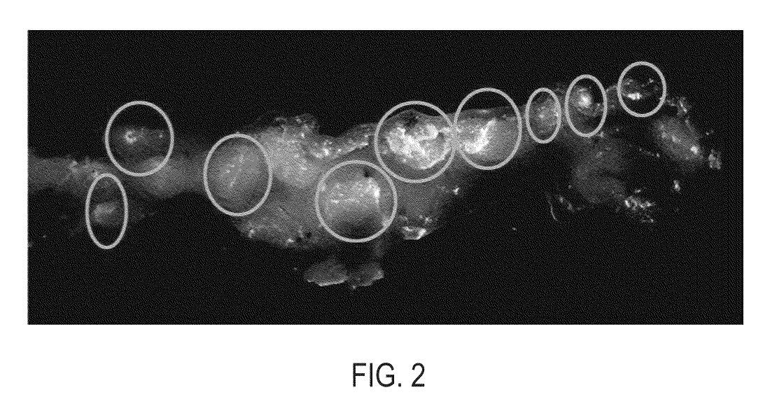

FIG. 2 shows the ex vivo Multispectral Optical Imaging (MSI) results of the APC^{Min/+} mouse tissues that were incubated in FITC-UEA-1. The ex vivo MSI results were consistent with the results of fluorescent microscopy UEA-1 histochemistry. The polyps are outlined. The intensity of lectin uptake by the polyps as compared with the normal tissue is visible. The ex vivo MSI showed uptake of the FITC-UEA-1 by the polyps. Over-expression of α-L-fucose by the polyps, versus the normal mucosa, was found.

FITC-UEA-1 confirmed the over-expression of a-L-fucose by the polyps on fluorescence microscopy in 17/17 cases, that is, 13 by histology and 4 by MSI.

### Example 3

### CT Detection Agent: UEA-1 Conjugated Polymerized Liposomes

In an embodiment, a computed tomography (CT) detection agent includes Ulex europaceus agglutinin 1 (UEA-1) conjugated to a liposome, with the liposome including a radiologic contrast agent. To produce the CT detection agent, total lipid in chloroform was dried to form a thin lipid film, hydrated with iodinated contrast solution, extruded and crosslinked to form polymerized liposomes. UEA-1 protein was then conjugated to the liposomes.

The polymerizable diacetylene phospholipid DAPC (Avanti Polar Lipids, Alabaster, Ala.) was mixed with the saturated spacer lipid DNPC (Lipoid, Newark, N.J.) and DMPE-NHS (NOF) with 1:1 ratio together with 1% 18:1 Lissamine Rhodamine PE (Avanti Polar Lipids). The lipids in chloroform were added to a round bottom flask in the dark and protected with aluminum foil. The solvent was removed under vacuum using BUCHI Rotavapor for 1-2 hrs and further removed by leaving the mixture under high vacuum for >24h. The concentrations of lipids were 20 μmol DAPC, 2 μmol DNPC, and 18 μmol DMPE-NHS and 0.4 μmol 18: Lissamine Rhodamine PE. The lipid film was hydrated with 10 mL (500 mmol, pH 8.8), pre-heated to 55° C., iodinated contrast solution (Diatrizoic acid sodium salt dihydrate, Sigma).

A 10 mL Lipex™ Thermobarrel Extruder (Northern Lipids Inc, Canada) was used to extrude the liposomes according to the manufacturer's instruction. A polycarbonate filter with 100 nm pore size was used. The extruder was placed in a 55° C. water bath so that the filter support base is below the water line, the liposomes were extruded for 10 cycles. The extruded liposomes were then polymerized on ice with a UV Stralinker 1800 (Stragene, La Jolla, Calif.) cross-linker for 20 cycles at 3600 μJ per cycle to form the polymerized liposomes.

For UEA-1 conjugation, polymerized liposomes were first changed to MES buffer using PD-10 column (Pierce) according to the manufacturer's instruction. 4 mg of EDC (Pierce) and 6 mg of sulfo-NHS (Pierce) was added to the liposomes for min at room temperature to recover a partly hydrolyzed NHS group. The liposomes were then changed to PBS using PD-10 column and 15 mg of the UEA-1 was added. The mixture was incubated at 4° C. shaker overnight. The conjugated polymerized liposomes were analyzed by column chromatography and transmission electron microscopy (TEM). The liposomes were stored at 4° C. until further use.

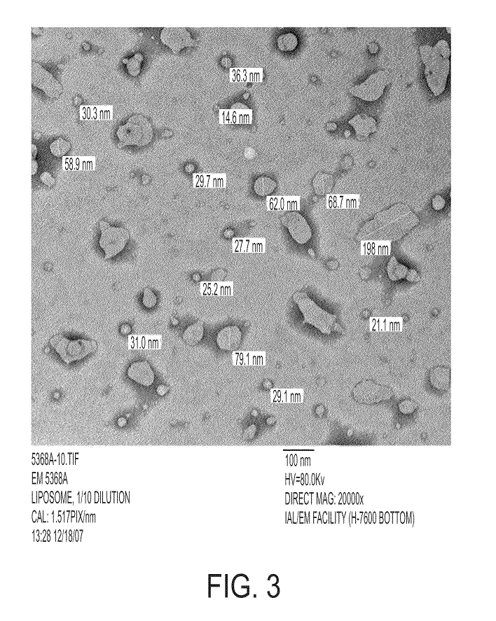

FIG. 3 shows the TEM images of the polymerized liposomes with a size range of 15 nm to 80 nm. The liposomes were mostly spherical with some non-uniformity in shape and size.

The CT detection agent produced thus had two imaging modalities. First, the radiographic contrast agent, diatrizoic acid sodium salt dihydrate, included in the liposomes, enabled detection of the CT detection agent by reducing or blocking the transmission of X-radiation transmitted through a sample containing the CT detection agent. Second, the Lissamine Rhodamine PE chromophore included in the liposomes enabled detection of the CT detection agent by irradiating the CT detection agent with light and detecting fluorescence of the chromophore.

### Example 4

### In Vitro Imaging of CT Detection agent in Colon Tissue

Three colons from the C57BL/6J APC^{Min/+} mouse model were excised and sectioned onto glass microscope slides in 10 μm slices using a cryostat. The sections were fixed in formaldehyde for 15 mins and incubated in serial dilutions of conjugated and nonconjugated liposomes at room temperature for 45 mins. The slides were washed in PBS, before being mounted and viewed by optical microscopy.

The CT detection agent used was polymerized liposomes conjugated with lectin UEA-1. Three colons from the C57BL/6J APC^{Min/+} mouse model were excised and sectioned onto glass microscope slides in 10 μm slices using a cryostat. The conjugated (with UEA-1 lectin) and non-conjugated (without UEA-1 lectin) liposomes were applied to the colon tissue.

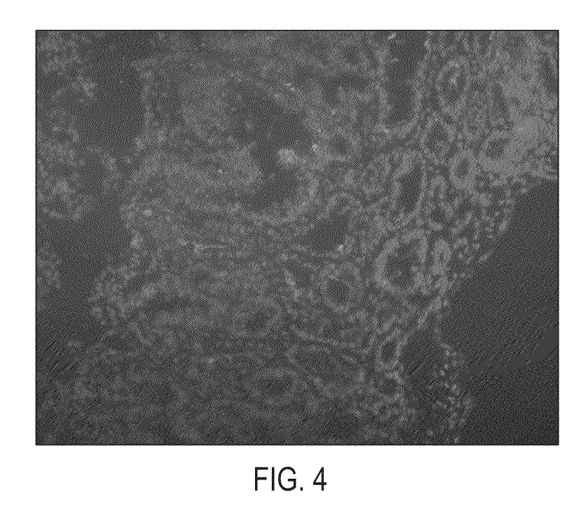

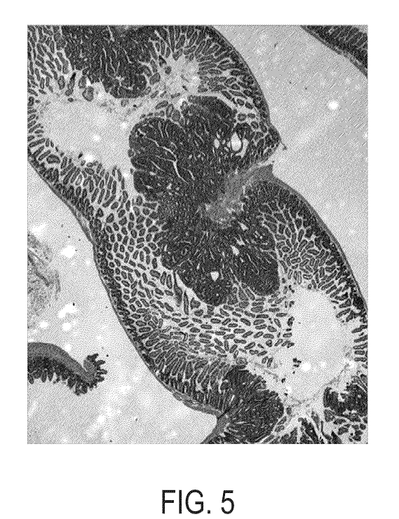

FIG. 4 presents the results of the imaging. The polyp is the massive structure which encompasses the majority of the viewing field. The red (brighter) color resulted from the rhodamine contrast agent on the surface of the UEA-1 conjugated liposomes. By contrast, the normal mucosa, represented by cells at the top left of the field of view, do not show much red (brighter) color, the UEA-1 conjugated liposomes did not exhibit a high concentration in the vicinity of this normal tissue. Thus, the CT detection agent, UEA-1 conjugated liposomes, preferentially bound to polyps in the APC^{Min/+} mouse colon tissue, which overexpresses the carbohydrate α-L-fucose, as compared with normal mucosa. These results suggest the use of UEA-1 conjugated liposomes for small animal detection of colorectal polyps and potentially also colorectal carcinoma.

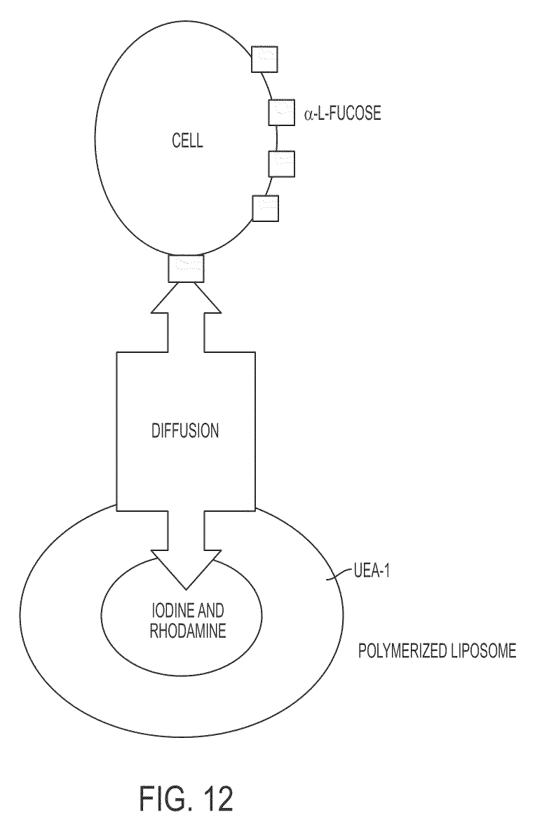

FIG. 12 presents a cartoon of an experimental design.

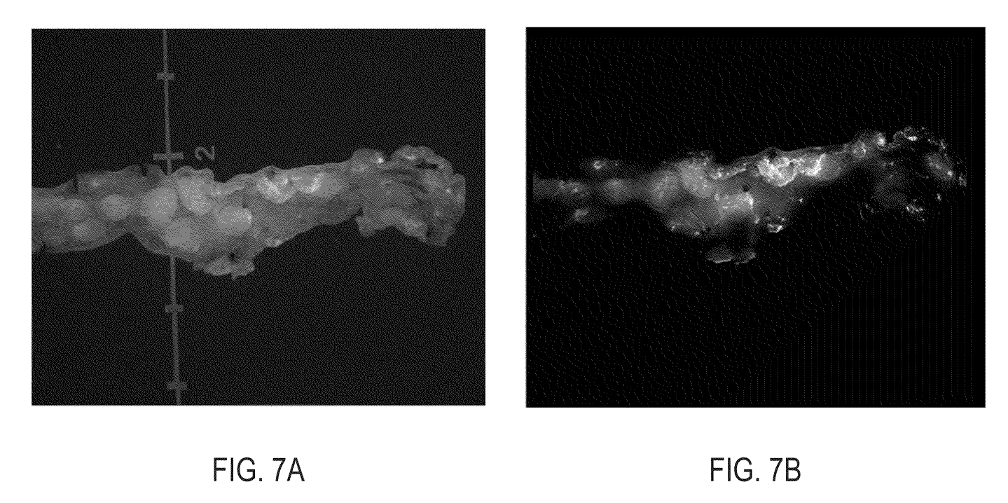

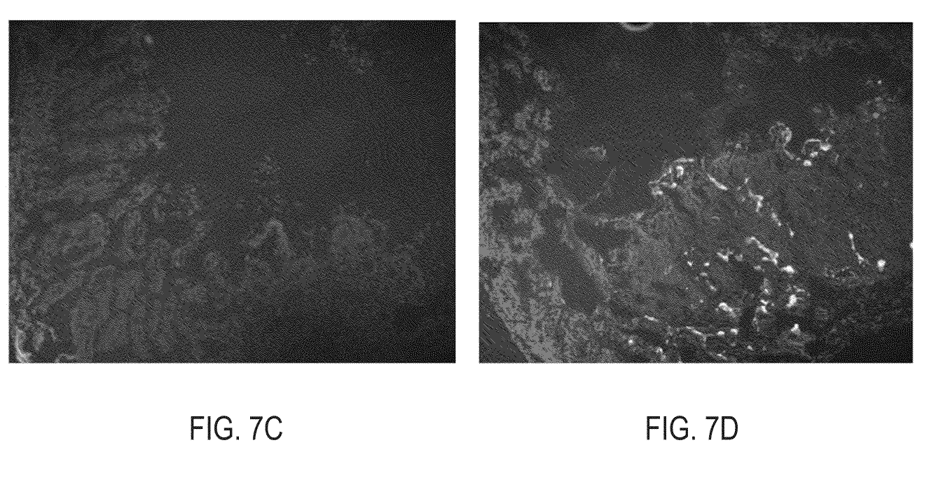

In an embodiment, imaging was performed at 10 nm intervals, from 500 nm to 720 nm, with 800 ms exposure. An optical imaging system can be used that includes an emission filter and a lens between a CCD camera and the specimen. The imaging can be conducted in a light proof chamber. An excitation light source fitted with an excitation filter can be used. FIGS. 7A and 7B show the results of ex vivo Multispectral Imaging (MSI) of APC^{Min/+} mouse colons in the presence of FITC-UEA-1. FIG. 7B shows the image with the background substracted. Protruding bumps visible in the images are polyps. FIG. 7D presents an image obtained by optical imaging of UEA-1 conjugated polymerized liposomes (targeted) that bind the surface of a polyp found in APC^{Min/+} tissue. FIG. 7C presents an image from a control experiment in which polymerized liposomes having no protein conjugation (nontargeted) are used. Thus, the UEA-1 targeted liposomes bind APC^{Min/+} mouse tissue with over-expression of α-L-fucose, whereas the nontargeted liposomes do not bind tissue.

In additional embodiments according to the present invention, techniques similar to those described above are used to identify other lectins that specifically target neoplastic tissue, and do not target normal tissue. For example, lectins other than UEA-1 that target neoplastic tissue that secretes carbohydrates other than α-L-fucose in excess can be identified. Carbohydrates other than α-L-fucose that are secreted in excess by neoplastic tissue in comparison with normal tissue can be identified, and lectins specific to such carbohydrates can be identified. Conversely, carbohydrates that are secreted in lesser quantity from neoplastic tissue in comparison with normal tissue can be identified, and lectins specific to such carbohydrates can be identified; such carbohydrates can be used to target normal tissue, so that the absence of detection agent functionalized with the carbohydrate is indicative of neoplastic tissue. Lectins that target neoplastic tissue other than adenomatous polyps can be identified. Lectins that target neoplasms in tissue other than the bowels, colon, or rectum can be identified. Thus, techniques similar to those described above can be used to target and identify neoplasms associated with cancers other than colorectal cancer and in parts of the body other than the bowels, colon, or rectum. The detection agent can include a polymerized liposome, a polymerized vesicle, and/or a polymer. The UEA-1 or other selective lectin or antibody can be conjugated to the liposome, vesicle, polymer, and/or imaging agent. The imaging agent can be absorbed within the liposome, vesicle, or polymer. The liposome or vesicle can be formed from one or more surfactants, for example, lipids, such as phospholipids.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. de la Chapelle, A., Genetic predisposition to colorectal cancer. Nat Rev Cancer, 2004. 4(10): p. 769-80.
2. Potter, J. D., Colorectal cancer: molecules and populations. J Natl Cancer Inst, 1999. 91(11): p. 916-32.
3. Lamlum, H., et al., The type of somatic mutation at APC in familial adenomatous polyposis is determined by the site of the germline mutation: a new facet to Knudson's ‘two-hit’ hypothesis. Nat Med, 1999. 5(9): p. 1071-5.
4. Goss, K. H. and J. Groden, Biology of the adenomatous polyposis coli tumor suppressor. J Clin Oncol, 2000. 18(9): p. 1967-79.
5. Kobaek-Larsen, M., et al., Review of colorectal cancer and its metastases in rodent models: comparative aspects with those in humans. Comp Med, 2000. 50(1): p. 16-26.
6. Lynch, H. T., et al., Familial cancer syndromes: a survey. Cancer, 1977. 39(4 Suppl): p. 1867-81.
7. Aaltonen, L. A., et al., Clues to the pathogenesis of familial colorectal cancer. Science, 1993. 260(5109): p. 812-6.
8. Aaltonen, L. A., et al., Replication errors in benign and malignant tumors from hereditary nonpolyposis colorectal cancer patients. Cancer Res, 1994. 54(7): p. 1645-8.
9. Fearon, E. R. and B. Vogelstein, A genetic model for colorectal tumorigenesis. Cell, 1990. 61(5): p. 759-67.
10. Osborn, N. K. and D. A. Ahlquist, Stool screening for colorectal cancer: molecular approaches. Gastroenterology, 2005. 128(1): p. 192-206.
11. Ahlquist, D. A. and J. A. Gilbert, Stool markers for colorectal cancer screening: future considerations. Dig Dis, 1996. 14(3): p. 132-44.
12. Levin, B., et al., Emerging technologies in screening for colorectal cancer: CT colonography, immunochemical fecal occult blood tests, and stool screening using molecular markers. CA Cancer J Clin, 2003. 53(1): p. 44-55.
13. Ahlquist, D. A., et al., Patterns of occult bleeding in asymptomatic colorectal cancer. Cancer, 1989. 63(9): p. 1826-30.
14. Limburg, P. J., et al., Prospective evaluation of fecal calprotectin as a screening biomarker for colorectal neoplasia. Am J Gastroenterol, 2003. 98(10): p. 2299-305.
15. Dubrow, R., C. S. Kim, and A. K. Eldred, Fecal lysozyme: an unreliable marker for colorectal cancer. Am J Gastroenterol, 1992. 87(5): p. 617-21.
16. Nakayama, T., et al., ELISA for occult faecal albumin. Lancet, 1987. 1(8546): p. 1368-9.
17. O'Connor, S. D., et al., Oral contrast adherence to polyps on CT colonography. J Comput Assist Tomogr, 2006. 30(1): p. 51-7.
18. Hollingsworth, M. A. and B. J. Swanson, Mucins in cancer: protection and control of the cell surface. Nat Rev Cancer, 2004. 4(1): p. 45-60.
19. Jass, J. R., et al., Ulex europaeus agglutinin-1 binding in hereditary bowel cancer. Pathology, 1993. 25(2): p. 114-9.
20. Ezpeleta, I., et al., Preparation of Ulex europaeus lectin-gliadin nanoparticle conjugates and their interaction with gastrointestinal mucus. Int J Pharm, 1999. 191(1): p. 25-32.
21. Boland, C. R., R. S. Bresalier, and J. A. Roberts, Assay of lectin binding sites in colon cancer-associated mucins: comparison of fluorescence microscopy with a quantitative chromatographic technique. J Histochem Cytochem, 1988. 36(10): p. 1329-34.
22. Kuroki, T., et al., Lectin staining of neoplastic and normal background colorectal mucosa in nonpolyposis and polyposis patients. Dis Colon Rectum, 1991. 34(8): p. 679-84.
23. Yonezawa, S., et al., Glycoconjugate with Ulex europaeus agglutinin-I-binding sites in normal mucosa, adenoma, and carcinoma of the human large bowel. J Natl Cancer Inst, 1982. 69(4): p. 777-85.
24. Yonezawa, S., et al., Binding of Ulex europaeus agglutinin-I in polyposis coli: comparative study with solitary adenoma in the sigmoid colon and rectum. J Natl Cancer Inst, 1983. 71(1): p. 19-24.
25. Brockhausen, I., Glycodynamics of mucin biosynthesis in gastrointestinal tumor cells. Adv Exp Med Biol, 2003. 535: p. 163-88.
26. Dahiya, R., et al., Mucin oligosaccharide biosynthesis in human colonic cancerous tissues and cell lines. Cancer, 1992. 70(6): p. 1467-76.
27. Kim, Y. S., R. Isaacs, and J. M. Perdomo, Alterations of membrane glycopeptides in human colonic adenocarcinoma. Proc Natl Acad Sci USA, 1974. 71(12): p. 4869-73.
28. Hakomori, S., Aberrant glycosylation in cancer cell membranes as focused on glycolipids: overview and perspectives. Cancer Res, 1985. 45(6): p. 2405-14.
29. Velcich, A., et al., Colorectal cancer in mice genetically deficient in the mucin Muc2. Science, 2002. 295(5560): p. 1726-9.
30. Otori, K., et al., Emergence of adenomatous aberrant cryptfoci (ACF) from hyperplastic ACF with concomitant increase in cell proliferation. Cancer Res, 1995. 55(21): p. 4743-6.
31. Limburg, P. J., et al., Immunodiscrimination of colorectal neoplasia using MUC1 antibodies: discrepant findings in tissue versus stool. Dig Dis Sci, 2000. 45(3): p. 494-9.
32. Lotan, R. and A. Raz, Lectins in cancer cells. Ann N Y Acad Sci, 1988. 551: p. 385-96; discussion 396-8.
33. Barondes, S. H., Soluble lectins: a new class of extracellular proteins. Science, 1984. 223(4642): p. 1259-64.
34. Watanabe, M., et al., Appearance of Ulex europaeus agglutinin-1 and Griffonia simplicifolia agglutinin-1 binding sites on cancer cells in sigmo-rectal polyps. Acta Pathol Jpn, 1992. 42(11): p. 800-6.
35. Ouyang, D. L., et al., Noninvasive testing for colorectal cancer: a review. Am J Gastroenterol, 2005. 100(6): p. 1393-403.
36. Atkin, W. and J. P. Martin, Stool DNA-based colorectal cancer detection: finding the needle in the haystack. J Natl Cancer Inst, 2001. 93(11): p. 798-9.
37. Shamsuddin, A. M., A simple mucus test for cancer screening. Anticancer Res, 1996. 16(4B): p. 2193-9.
38. Hoskins, L. C. and E. T. Boulding, Mucin degradation in human colon ecosystems. Evidence for the existence and role of bacterial subpopulations producing glycosidases as extracellular enzymes. J Clin Invest, 1981. 67(1): p. 163-72.
39. Loktionov, A., et al., Quantitation of DNA from exfoliated colonocytes isolated from human stool surface as a novel noninvasive screening test for colorectal cancer. Clin Cancer Res, 1998. 4(2): p. 337-42.
40. Ahlquist, D. A., et al., Morphometric analysis of the “mucocellular layer” overlying colorectal cancer and normal mucosa: relevance to exfoliation and stool screening. Hum Pathol, 2000. 31(1): p. 51-7.
41. Ahlquist, D. A., et al., Colorectal cancer screening by detection of altered human DNA in stool: feasibility of a multitarget assay panel. Gastroenterology, 2000. 119(5): p. 1219-27.
42. Boynton, K. A., et al., DNA integrity as a potential marker for stool-based detection of colorectal cancer. Clin Chem, 2003. 49(7): p. 1058-65.
43. Dong, S. M., et al., Detecting colorectal cancer in stool with the use of multiple genetic targets. J Natl Cancer Inst, 2001. 93(11): p. 858-65.
44. Frentz, S. M. and Summers, R. M., Current Status of CT Colonography. Acad. Radiol., 2006. 13: p. 1517-31.
45. Alonso-Romanowski S., Chiaramoni N. S., Lioy V. S., Gargini R. A., Viera L. I., Taira M. C., Chemistry and Physics of Lipids, 2003. 122: p. 191-203.

We claim:

1. A method, comprising:
providing a detection agent comprising
a polymerized liposome or polymer,
a UEA-1 or another selective lectin or an antibody, and
an imaging agent,
wherein the UEA-1 or other selective lectin or antibody is conjugated to the liposome or polymer or to the imaging agent,
wherein the imaging agent is absorbed in the liposome or polymer;
administering the detection agent to a section of lower gastrointestinal tract of an animal;
permitting the UEA-1 or other selective lectin to bind to carbohydrate or glycoprotein or antibody to bind to antigen expressed by a gastrointestinal polyp or neoplasm;
imaging the section of lower gastrointestinal tract; and
identifying regions with elevated concentrations of the imaging agent.

2. The method of claim 1, wherein the imaging comprises applying colonoscopy.

3. The method of claim 1, wherein the imaging comprises applying colonography using CT or MRI.

4. The method of claim 3, wherein applying colonography comprises selecting and applying a technique from the group consisting of bowel cleansing, gastrointestinal tract insufflation, performing scans to acquire images of the colon with the animal in multiple positions, altering the animal's position, obtaining thin tomographic slices with CT and/or MRI, optimizing the factors of decreased scanning time, decreased radiation exposure, and/or increased image quality, applying one or more image processing techniques, using two dimensional imaging in conjunction with three-dimensional imaging, using translucency rendering, applying a computer-aided detection (CAD) system, applying a CAD system that classifies a surface based on its local shape by using a geometric curvature parameter, making use of textural features and/or attenuation, applying filtering techniques, applying smoothing techniques, and/or applying an image processing technique, applying a CAD system that includes an improved display technique, applying a CAD system that segments the colon and generates colonic surface files in conjunction with a three-dimensional endoluminal viewer that uses the surface files in combination with images from a CT scanner to visualize anatomical landmarks and features such as polyps within the colon, applying a positioning technique to identify longitudinal and circumferential positions in the gastrointestinal tract, applying a registration technique to register datasets obtained with the subject in two or more positions and/or obtained at two or more different times, and combinations of these.

5. The method of claim 1, wherein the UEA-1 or other selective lectin selectively binds to a carbohydrate or glycoprotein or the antibody selectively binds to an antigen overexpressed by a gastrointestinal polyp or neoplasm in comparison to expression by normal gastrointestinal tissue.

6. The method of claim 1, wherein the selective lectin is Ulex europaeus agglutinin (UEA-1).

7. The method of claim 1, wherein the selective lectin is selected from the group consisting of Wheat germ agglutinin (WGA), Dolichos biflorus agglutinin (DBA), and Soybean agglutinin (SBA).

8. The method of claim 1, wherein the carbohydrate is α-L-fucose.

9. The method of claim 1, wherein the glycoprotein is selected from the group consisting of a mucin, a mucin secreted by a gastrointestinal polyp, and a mucin secreted by a gastrointestinal neoplasm, and combinations.

10. The method of claim 1, wherein the glycoprotein is a mucin secreted by a gastrointestinal polyp or neoplasm and not by normal tissue.

11. The method of claim 1, wherein imaging the section of lower gastrointestinal tract comprises
directing radiation to pass through the section of lower gastrointestinal tract of the animal and illuminate the imaging agent;
detecting radiation transmitted past, reflected by, emitted by, or modified by the imaging agent with a detector;
obtaining a radiograph from the detector;
using the radiograph to develop a map of concentration of imaging agent; and
using the map of concentration of imaging agent to develop a map of concentration of the carbohydrate and/or glycoprotein.

12. The method of claim 11, further comprising using the map of concentration of carbohydrate or glycoprotein to develop a map of gastrointestinal polyps or neoplasms in the section of lower gastrointestinal tract.

13. The method of claim 12, wherein the gastrointestinal polyps are adenomatous polyps.

14. The method of claim 11, further comprising using the map of concentration of carbohydrate or glycoprotein to determine whether the section of lower gastrointestinal tract comprises a gastrointestinal polyp or neoplasm.

15. The method of claim 14, wherein the neoplasm is selected from the group consisting of polyposis coli, adenoma, and adenocarcinoma.

16. The method of claim 11, further comprising using the map of concentration of carbohydrate and/or glycoprotein to determine whether the section of lower gastrointestinal tract comprises a carcinoma.

17. The method of claim 1, wherein the imaging agent is selected from the group consisting of a radiologic contrast agent, diatrizoic acid sodium salt dihydrate, an iodine-containing agent, a barium-containing agent, a fluorescent imaging agent, Lissamine Rhodamine PE, a stain, a dye, a radioisotope, a metal, a ferromagnetic compound, a paramagnetic compound, gadolinium, a superparamagnetic compound, iron oxide, a diamagnetic compound, and barium sulfate.

18. The method of claim 1, wherein the section of lower gastrointestinal tract is the colorectum.

19. The method of claim 1, wherein the animal is selected from the group consisting of a human, a small mammal, and a rodent.

20. The method of claim 1, wherein the detection agent comprises at least two imaging agents.

21. The method of claim 1, wherein the imaging agent comprises a radiologic contrast agent and
directing X-radiation to pass through the section of lower gastrointestinal tract of the animal and illuminate the radiologic contrast agent;
detecting X-radiation transmitted past the radiologic contrast agent with the detector;
obtaining a radiograph from the detector;
using the radiograph to develop a map of concentration of radiologic contrast agent;
using the map of concentration of radiologic contrast agent to develop a map of concentration of the carbohydrate or glycoprotein.

22. The method of claim 1, wherein the imaging agent comprises a fluorescing imaging agent and directing light to pass through the section of lower gastrointestinal tract of the animal and illuminate the fluorescing imaging agent;
detecting light emitted by the fluorescing imaging agent with the detector;
obtaining an optical image from the detector;
using the optical image to develop a map of concentration of fluorescent imaging agent; and
using the map of concentration of fluorescent imaging agent to develop a map of concentration of the carbohydrate or glycoprotein.

23. The method of claim 1, wherein the detection agent is administered orally or rectally.

24. The method of claim 1, wherein the detection agent is administered in a pill, tablet, capsule, or other conveniently orally ingestible format.

25. A method, comprising:
providing a detection agent comprising
a UEA-1 or another selective lectin or an antibody and
an imaging agent,
wherein the UEA-1 or other selective lectin or antibody is conjugated to the imaging agent,
administering the detection agent to a section of lower gastrointestinal tract of an animal;
permitting the UEA-1 or other selective lectin to bind to carbohydrate or glycoprotein or antibody to bind to antigen expressed by a gastrointestinal polyp or neoplasm;
imaging the section of lower gastrointestinal tract; and
identifying regions with elevated concentrations of the imaging agent.

26. A detection agent, comprising:
a polymerized liposome or polymer,
a UEA-1 or another selective lectin or an antibody, and
an imaging agent,
wherein the UEA-1 or other selective lectin or antibody is conjugated to the liposome or polymer or to the imaging agent.

27. The detection agent of claim 26, wherein the selective lectin is Ulex europaeus agglutinin (UEA-1).

28. A detection agent, comprising:
a UEA-1 or another selective lectin or an antibody and
an imaging agent,
wherein the UEA-1 or other selective lectin or antibody is conjugated to the imaging agent.
